(19) United States
(12) Patent Application Publication
(10) Pub. No.: US 2021/0259806 A1
(43) Pub. Date: Aug. 26, 2021

(54) ORTHODONTIC APPLIANCE FOR SELF-MONITORING OF TREATMENT

(71) Applicant: ForYouFirst AG, Vaduz (LI)

(72) Inventors: Alain KOUNGA, Dietikon (CH); Christoph APPERT, Vaduz (LI); Pedro GONÇALVES, Préverenges Vaud (CH)

(21) Appl. No.: 16/801,851

(22) Filed: Feb. 26, 2020

(51) Int. Cl. A61C 7/00; A61C 7/08; A61C 19/04; A61B 5/00

(52) U.S. Cl. CPC A61C 7/002; A61C 7/08; A61C 2201/002; A61B 5/4833; A61B 5/0022; A61C 19/04

(57) ABSTRACT

The present invention relates to a process for assessing stress relaxation in a dental arch-appliance orthodontic system. The information about stress relaxation in the clear aligner-arch system helps monitor the orthodontic treatment progress translated in teeth displacements and improve both patient's treatment compliance and timely treatment completion. The present invention relates to the efficacy of clear aligners by measuring or displaying the magnitude of residual stresses in the arch-aligner system. The measured magnitude of residual is indicative of the teeth displacement and the appropriate time to move to the next stage of the aligner treatment plan.

The present invention discloses a high esthetical orthodontic appliance which enables the treatment of malocclusion, teeth misalignment, crowding, overbite and underbite etc. The said appliance is manufactured such as to exhibit simple and powerful features for mobile health (mHealth), defined as the delivery of healthcare and healthcare supply through mobile devices. The said orthodontic appliance paired with an App on smartphones and tablets allows for a better monitoring of the orthodontic treatment. The invention further relates to a removable orthodontic appliance, having at least one reference marker applied thereto, wherein the at least one reference marker comprises a mechanochromic material. The invention relates to a set for aligning teeth with a removable orthodontic appliance, wherein the set comprises a plurality of removable orthodontic appliances having at least one reference marker applied thereto, wherein the at least one reference marker comprises a mechanochromic; or at least one removable orthodontic appliance and at least one reference marker comprising a mechanochromic material. The invention discloses a non-transitory computer readable medium, having a computer program logic recorded thereon which when executed by a processor causes a device to receive data corresponding to at least one image taken by a portable device for image acquisition and to compare the received data corresponding to the at least one image to reference data corresponding to a reference image.

A pleasing smile makes a great first impression and helps build self-esteem and self-confidence. Poorly aligned teeth are more difficult to brush and floss effectively, increasing the risk of tooth decay and periodontal (gum) disease, which can be painful and, without appropriate treatment, can lead to tooth loss. A key element of a great smile is good tooth alignment, which doesn't always happen naturally. How permanent teeth emerge depend on a number of factors; some are hereditary and others can be avoidable. Orthodontists develop solutions for re-arranging or solving tooth alignment problem and consequently improving the appearance of a person's teeth, smile, lower facial structure and profile. Beside cosmetic reasons the re-alignment can also aim at restoring functionality e.g. improving biting and chewing, help address speech problems, prevent abnormal wear of teeth and/or jaw joint pain, and make it easier to keep teeth and gums clean and healthy.

Aligners, braces and other removable appliances are among the most commonly used corrective tools. In certain situations, oral surgery may be recommended. For those who wish to be more discrete about the fact that they are undergoing orthodontic treatment, clear aligners or braces featuring brackets made out of a material that blends into the tooth color, are another state-of-the-art option. They look and work like metal braces, except the only component visible is the thin metal arch wire. Ceramic is not as durable as metal and can fracture under some types of stresses so they may not be the optimal solution in certain complicated cases. They typically cost more than their full-metal counterparts as well.

Invisalign® clear aligners are a series of clear plastic trays—each pair (top and bottom) slightly different than the preceding one—that fit over teeth and incrementally direct them into their desired positions. Those aligners can be removed temporarily for eating, brushing, flossing and special occasions. Being able to take clear aligners out whenever wanted, is a great advantage for the patient, but it's a significant disadvantage for a speedy case treatment. Since no progress can be made unless aligners are worn as prescribed (ideally 22 hours per day), the orthodontist must rely on the patient's motivation, compliance and dependability for the success of the treatment. Usually, every two weeks, a new aligner tray will be worn to encourage teeth into their projected final position. Most treatments will involve between 18-36 different aligners, but the actual duration of treatment will vary on the complexity of the teeth movement. Patient's motivation is key to treatment progress and adversely affected by the multiple time-consuming visits to the orthodontist who can assess the treatment status. Moreover, for different reasons patients are very busy throughout the day and find it cumbersome accommodating appointments for visits at the orthodontists, thus resulting in a poor monitoring of the treatment or even not considering an orthodontic treatment at all.

In many areas of healthcare delivery, electronic health (eHealth) is being examined for its promise to increase the overall efficiency of a healthcare system and to improve patient outcomes. As eHealth grows in scope and maturity, its potential includes improvements and enhancements to patient safety, health outcomes, financial efficiencies, and communication between multiple healthcare providers. The potential to share data over cellular and Wi-Fi networks within and across facilities, for expert consultation and data sharing among multiple care-givers, whether they are located in close proximity or separated by hundreds of miles, reduces the need to transport patients for specialized consultation, avoiding considerable patient physical and emotional stress, time, and expense.

Having recognized that patient's discipline and motivation are key factors for a speedy and successful brace-based orthodontic treatment, several inventors have filed patents disclosing solutions which comprise clear braces or trays equipped with active or passive sensors as well as actuators. U.S. 8,771,149 B2 discloses an orthodontic appliance equipped with a sensor that senses when the appliance is properly positioned and a processor that processes an output of the sensor and generates compliance data. In U.S. Pat. No. 8,899,976 B2 presents an orthodontic apparatus that includes an appliance for placement over one or more teeth and a release agent receptacle including an outer portion provided on appliance surface, an inner portion provided within the outer portion and one more apertures formed in the outer portion. The apparatus is equally conceived for treatment compliance monitoring. In EP2164422A1 treatment progress is tracked by matching teeth from a previously segmented model to a surface of an unsegmented representation of the actual arrangement and calculating one or more positional differences between the actual and planned arrangements. US2009117507A1 discloses an apparatus for monitoring orthodontic treatment compliance, including an appliance adapted to be worn over one or more teeth; and a compliance indicator mounted on the appliance.

In US2016066776 A1 an oral monitoring system is provided. The oral monitoring system includes one or more cameras configured for installation in a mouth for capturing images. A memory is configured for storing images. One or more processors are configured to process images captured by the one or more cameras, and a wireless transmitter is configured to transmit data corresponding to the captured images. A chemical sensor is further provided and the wireless transmitter is further configured to transmit data corresponding to substances detected by the chemical sensor. WO2016116874 A1 provides the invention of an improved orthodontic bracket with integrated 30 piezo-sensor chip determining even the slightest change in initial installation setup of orthodontic bracket system and farther notifying the subscriber electronically. The invention provides a detachable wireless frequency transmitter to be placed on the bracket of tie-wing to permit clinical recycling of the bracket; and optionally allowing a handheld laser scanner/photospectrometer to measure the frictional coefficient between the arch-wire and bracket to determine exact amount of force required for tooth retraction; to determine the bonding technique for detecting voids in adhesive; and to determine the distance between forces applied and center of mass of the tooth. In U.S. 6,890,285 B2, a brace compliance monitor that includes a compliance sensor, a signal processor, and a display is disclosed. Compliance data is displayed on the display to provide the patient or subject with immediate compliance information on whether they have been wearing the brace for the specified period and in the specified manner. The brace compliance monitor may also include a secondary sensor such as a tilt sensor, a pressure sensor, a force sensor, an acceleration sensor, or a velocity sensor. The secondary sensors may provide additional compliance data to the patient and health care provider.

According to US2013140289A1, a compliance monitoring system for an intraoral appliance comprises a power source, a detector for detecting when the intraoral appliance is positioned in the mouth for use, a recorder configured to record measurement data, and a transponder configured to communicate the measurement data are presented in the patent application. The monitoring system is adjustable based on a particular property of a patient or a group of patients.

Bradley Donald has published in WO2013/075246A1 a dental appliance therapy compliance monitoring apparatus that includes a battery to power the apparatus, a_temperature sensor, a spatial orientation sensor, a processor configured to control the temperature sensor and the spatial orientation sensor to periodically measure the ambient temperature and the spatial orientation to obtain a time-domain series of ambient temperature measurement and a time-domain series of spatial orientation measurements, respectively; a memory-operatively coupled to the processor to record the ambient temperature measurements and spatial orientation measurements, and a communication module coupled operatively to the processor. The compliance verification processor is configured to communicate the recorded time-domain series of ambient temperature measurements and the time-domain series of spatial orientation measurements and to determine compliance with the dental appliance therapy.

There is therefore a need for removable orthodontic appliances, sets for aligning teeth with a removable orthodontic appliances, a non-transitory computer readable medium, having a computer program logic recorded thereon which when executed by a processor causes a device to process data corresponding to at least one image taken by a portable device for image acquisition, and a method for aligning teeth by means of removable orthodontic appliances and for monitoring a treatment progress which is reliable, ensures discipline of the patient and enables monitoring throughout the treatment. It is a purpose of the present invention to overcome the disadvantages comprised in the state of the art. In particular, it is a purpose of the present invention to provide an accurate method of monitoring and quantifying the therapy progress on a regular basis.

### DESCRIPTION OF THE INVENTION

The problem is solved by a removable orthodontic appliance, a set for aligning teeth, a non-transitory computer readable medium, having a computer program logic recorded thereon, a system and a method according to the independent claims.

The invention relates to a method for aligning teeth by means of removable orthodontic appliances and for monitoring a treatment progress, the method comprising the steps of:
providing a series of removable orthodontic appliances for incrementally directing the teeth into a desired teeth arrangement;
applying reference points to the removable orthodontic appliances;
receiving data corresponding to at least one image taken by a portable device for image acquisition, wherein the at least one image depicts the removable orthodontic appliance;
comparing the received data corresponding to the at least one image to reference data corresponding to a reference image.

The method is preferably performed repeatedly for consecutive treatment steps of a multi-step orthodontic treatment.

A process for aligning teeth with removable orthodontic appliances is known in the art. It has been described for example in application No. U.S. Pat. No. 5,975,893 A, the contents of which are included herein by reference. According to the process of repositioning teeth with clear dental aligners described by Chishti et al., patent teeth are repositioned from an initial tooth arrangement to a final tooth arrangement by placing a series of incremental position adjustment appliances in the patient's mouth. Conveniently the appliances are not affixed and the patient may place and replace the appliances at any time during the procedure configured to receive the teeth in a cavity and incrementally reposition individual teeth in a series of at least three. The first appliance of the series will have a geometry selected to reposition the teeth from the initial tooth arrangement to a first intermediate arrangement. After the first intermediate arrangement is approached or achieved, one or more additional (intermediate) intermediate appliances will be successively placed on the teeth, where such additional appliances have geometries selected to progressively reposition the teeth from the first intermediate arrangement through successive intermediate arrangement(s). The treatment will be finished by placing a final appliance in the patient's mouth, where the final appliance has a geometry selected to progressively reposition teeth from the last intermediate arrangement to the final tooth arrangement.

In general, the transition to the next appliance can be initialized based on a number of factors. Most simply, the appliances can be replaced on a predetermined schedule or at a fixed time interval (i.e. number of days for each appliance) determined at the outset based on an expected or typical patient response. Alternatively, actual patient response can be taken into account, e.g. a patient can advance to the next appliance when that patient no longer perceives pressure on their teeth from a current appliance, i.e. the appliance they have been wearing fits easily over the patient's teeth and the patient experiences little or no pressure or discomfort on his teeth.

However, determining the appropriate moment for proceeding to the next orthodontic appliance can be difficult or patients may experience some discomfort during the procedure. In this situation patient's motivation is highly needed. In order to prevent a patient from interrupting or abandoning the treatment, a way of accurately quantifying the treatment progress while adjusting the pressure exerted on the teeth can be of great importance.

By using a process for aligning teeth and by accompanying the said process with a series of monitoring steps as described above, stress control and patient compliance can be significantly enhanced.

The term “removable orthodontic appliances” includes in particular arch-appliance assemblies tor modeling a dental impression of a patient with or without repositioning geometries in order to shift the dental situation towards a desirable state. The removable orthodontic appliances are in particular arch-appliance assemblies, but include also other systems or assemblies. They are typically plastic trays, preferably transparent plastic trays.

It is an advantage of the claimed method that smartphones and tablet cameras, which are ubiquitously used for recreational photo capture and sharing, can be used for monitoring purposes. In case the built-in cameras are optimized for small size, they can generally leverage the growth of mobile health (mHealth) and support patient's motivation. The latter is a key aspect of a successful orthodontic treatment.

The process is preferably such that at least one of an alignment and a positioning of the reference points is used to assess stress relaxation in the orthodontic appliances. As described above, a regular, preferably daily, verification of residual strain in the orthodontic appliance in use provides for both tolerable pressure on the patient's jaw and motivational feedback regarding therapy progress. With such an arrangement, motivation and discipline of the patient as well as monitoring throughout the treatment can be realized.

The manufacturing processes for medical plastics, including clear plastic for transparent aligners, such as injection molding, extrusion, vacuum forming and machining, inherently generate internal stresses. These stresses sometimes have an intentional and highly desirable purpose. However, typical effects of internal stresses also include: Distortion due to stress relaxation, cracks, deterioration of optical performance, changes in mechanical properties. Internal stresses become real loads applied to the material upon exposure of the part to slightly elevated temperatures.

When using orthodontic appliances as described above, trays fit tightly to the teeth arches when mounted into the patient's mouth. This arch-appliance assembly features some internal stresses ruled by Newton's third law (actio=reactio). That is, when 2 bodies are in contact, the forces applied by one on another are always equal in magnitude and opposite in direction. As the appliances are slightly smaller than the jaw and made of a material with high tensile modulus of up to E=2000 MPa they exhibit a strain when mounted on the jaws. Based on Hooke's law σ=E·ε, where σ is the stress in the material and ε represents the corresponding strain, it is evident that in the case of small stresses (elastic region of Hooke's law) a stress reduction or increase is directly translated into a decrease or increase of strain and equally translated into the displacement of teeth. Teeth Movement will lead to stress relaxation or decrease in the appliance material. Thus, the related variation in strain of the appliance's material can be regarded as a good measure of the distance moved by teeth. In the present invention it is proposed to use this indicator to permanently monitor the progress of the therapy.

It is preferred that prior to the image acquisition, a magnifying lens is attached to a camera of the portable device for high resolution image acquisition of orthodontic appliances mounted on dental arches such that stress relaxation in the orthodontic appliances can be visualized.

Since the appliance's material features a high tensile modulus and the stresses generated for teeth movement are small, the resulting strain is small as well. Measuring those small distances is improved by using a magnifying lens which is attached to the camera of a smart phone. Therapy progress is then assessed by comparing two consecutive images of the appliance mounted on the arch.

It is preferred that the acquired images are super-imposed and an image comparison tool is used to extract information about at least one of
a stress relaxation in the orthodontic appliances; and
a displacement of the patient's teeth.

A pixel by pixel image comparison tool, such as a software, will help to determine changes in the shape of the reference structure. The changes will be correlated to stress relaxation in the arch-appliance assembly and thus indicative of the teeth displacement. Thus, the patient can easily monitor the movement progress and can transfer immediately the results to the treating orthodontist or dental practitioner for review. Based on the results of the image comparison, both the patient and the treating dental practitioner can easily make decisions with regard to the appropriate time to change the aligners and move to the next step of the treatment. The results of the image comparison are hence used as a monitoring tool for an orthodontic treatment.

In a preferred embodiment, images are acquired by means of lenses configured to visualize residual stresses in the orthodontic appliances based on birefringence. The visualization serves as a basis for qualitative or quantitative measurements of residual stresses or of stress release. Graphic printed objects can be used as reference points to facilitate at least one of a measurement or a visualization of a residual stress or a stress release. The graphic elements may be designed such that detection by eye is rendered possible may be designed such that they are only visible under light other than white light and thus do not compromise the aesthetics of the teeth and the orthodontic appliances. For example, the graphic elements may be designed such that they are only visible under UV-light.

Suitable reference points or stress state indicators can be chosen according to the specific needs of the patient or the envisaged treatment. Visible graphic elements might be economic and simple in use while reference points not visible to the naked eye may accommodate for higher aesthetic requirements.

In one embodiment of the invention, a reference aligner can be used for measuring the teeth displacement, wherein the reference aligner does not displace the teeth.

Another aspect of the invention relates to a method, where the orthodontic appliances have at least one element of the group of sensors and actuators attached or included thereto, wherein the element is configured to communicate with a patient's portable device. This method can form an aspect of the method as described above or can be used as an independent method.

Ideally sensors and/or actuators attached or included in the braces communicate with a patient's portable device. An instant feedback on a patient's portable device, such as a smartphone, a handheld PDA, a computer, etc. has proven highly beneficial. The communication may trigger the portable device to remind the patient of wearing the braces (disciplinary aspect) and/or to inform the patient about the treatment's progress (monitoring and motivational aspect).

For structural health monitoring the inventor proposes for example RFID sensors or actuators. For example, RFID strain gauge sensors need no wires or batteries, hence no maintenance, and ensure a long sensor's life. They can be read through materials that typically cover structural components, with read range up to several meters on a metal-mount sensor and data logging capability. Each wireless strain gauge sensor has a unique ID number so that many sensors can be read by just one reader at the time. Moreover, a Bluetooth-connection can equally be considered for data transfer between a brace and a portable device.

One aspect of the invention relates to a method as described above, wherein a calibration device configured to standardize the displacement measurements is mounted on the aligner.

One aspect of the invention relates to a method, wherein the calibration device is adjusted by a user, e.g. a patient, to monitor changes of the tooth position.

In terms of progress monitoring an aligner's set can be provided with appliances, preferably at least two appliances, very similar to a mouthguard used by athletes in sports e.g. rugby, boxing etc. In this case, the method as claimed above further includes a step prior to the teeth alignment treatment or following a treatment step. This step consists of using a set of sports mouthguard-like appliances to mold a patient's teeth arrangement, therefore enabling a patient to self-monitor an orthodontic treatment progress. The monitoring is achieved by comparing an initial mold with molds taken at later steps of the treatment. This method can form an aspect of the method as described above or can be used as an independent method.

The mouthguard material is very pliable and soft when placed in hot water so it can be molded to a perfect fit and then solidifies into a perfect customised mold of the teeth. Putting this back in to hot water will delete any impression stored in the material. Thus, the first mouthguard, (called “mold 1”) will be used by the patient before the first use of aligners and will be regarded and kept as reference/mold of the teeth at the beginning of the treatment. In the course of the treatment, the second mouthguard (called “mold 2”) will be used by the patient for repeated impression-takings and so assessing how the teeth position changes incrementally at subsequent stages of the treatment. The treatment progress is then evaluated by comparing the impression in “mold 1” and that of “mold 2”. The assessment can be performed by the patient at home, independently of the assistance of a dental practitioner.

Furthermore, the problem is solved by a removable orthodontic appliance, having at least one reference marker applied thereto, wherein the at least one reference marker comprises a mechanochromic material. It is preferred that the removable orthodontic appliance comprises a plurality of markers of mechanochromic material.

The removable orthodontic appliance is particularly suitable for being used in a method for aligning teeth as described above.

The term “mechanochromic material” refers to a material which changes its optical properties (e.g. its color, conformation or refractive index) according to mechanical conditions, preferably stress. The stress level may be either a pressure or a strain. It is preferred that the mechanochromic material is a piezochromic material. In this embodiment, the aligning tool (i.e. a clear aligner or bracket) may be covered partially with a piezochromic coating.

The use of a piezochromic material facilitates visualization and/or measurement of a residual stress in a removable orthodontic appliance. The piezochromic coating undergoes a visible color change when the internal stress state in the system (of aligning tool—teeth) falls below a defined level, known to no longer effect the displacement of the teeth. The piezochromic material can be deposited as coating or embedded in a composite matrix. When embedded in a composite the piezochromic can also be used as a clear indicator of the proper positioning of the aligning appliance. In an embodiment, where the measuring or monitoring of a stress level in the orthodontic appliances occurs by means of a piezochromic material, a piezochromic coating or a piezochromic ink embedded in a matrix can be used as a pressure sensor or differential pressure sensor in the appliance.

The at least one reference marker applied to the removable orthodontic appliance can be a graphic printed element. In this regard, a reference (e.g. a cross “X”, a rectangle, a circle, a “V”, or marks similar to those used for attachments buttons, etc ..) is inscribed in the appliance at position, e.g. in the anterior and visible region of the dentition.

In one embodiment, the graphic printed element can be designed such that detection by eye is rendered possible. In an alternative embodiment, the graphic printed element is only visible under light other than white light. Such a graphic printed element does not compromise the aesthetics of the teeth and the orthodontic appliances.

The mechanochromic material can be an organic chromophore dispersed in polymer matrix, an inorganic solid, or a liquid crystal element. For example, the mechanochromic material can be selected from the group of 1,4-bis(α-cyano-4-methoxystyryl)benzene or 1,4-bis(α-cyano-4-methoxystyryl)-2,5-dimethoxybenzene in linear low-density polyethylene; 1,4′-bis(2-benzoxazolyl)-stilbene in polypropylene; or diaryl-bi-benzofuranone mechanophores embedded in polyurethanes. Other suitable mechanochromic materials include be MaSrCaMgSi₂O₇:Eu²⁺ and SrBaMgSi₂O₇:Eu²⁺. Further suitable pressure indicating materials are crystals of toluene sulphonate diacetylene polymers, co-polymers containing poly-diacetylenes or poly-silylenes. Alternatively, the pressure indicating material may be one susceptible to relative changes in refractive index upon the application of pressure, for example aromatic solvents containing poly-(N-methyl-acrylamide).

It is preferred that orthodontic appliances have a calibration device mounted thereto which is configured to standardize the displacement measurements. It is preferred that the said calibration device can be adjusted by a user to monitor changes of a tooth position.

It is preferred that the removable orthodontic appliance according to claim 1 has at least one element of the group of sensors and actuators attached or included thereto, wherein the element is configured to communicate with a patient's portable device. Such configuration further improves efficient performance of a method as described above.

The invention further relates to a set for aligning teeth with a removable orthodontic appliance, wherein the set comprises:
a plurality of removable orthodontic appliances having at least one reference marker applied thereto, wherein the at least one reference marker comprises a mechanochromic material; or
at least one removable orthodontic appliance and at least one reference marker comprising a mechanochromic material.

The set is particularly suitable for being used in a method for aligning teeth as described above. It is also preferred that the marker and the mechanochromic material are configured as described above.

In one embodiment, the set comprises a plurality of removable orthodontic appliances having at least one reference marker applied thereto, wherein the at least one reference marker comprises a mechanochromic material. The removable orthodontic appliances are configured as a series of appliances. The series of appliances repositions a patient's dental situation from an initial tooth arrangement to a final tooth arrangement by placing the series of incremental position adjustment appliances consecutively in the patient's mouth. In a particularly preferred embodiment, fixed references are positioned in the appliance's area covering the gingiva.

In another embodiment where the set comprises at least one removable orthodontic appliance and at least one reference marker comprising a mechanochromic material, it is preferred that the set comprises a series of removable orthodontic appliances configured to reposition a patient's dental situation from an initial tooth arrangement to a final tooth arrangement by placing the series of incremental position adjustment appliances consecutively in the patient's mouth. The reference marker(s) and the mechanochromic material are preferably configured as described above. However, in this case the reference marker(s) of mechanochromic material are applicable to a tooth instead of the orthodontic appliance(s). In a preferred setup the references are put on critical teeth of the anterior region. In a preferred embodiment, the mark or reference spreads across at least 4 teeth in the anterior region and most preferably 8 teeth, thus emulating the position of centric occlusion of the patient. It is preferred that the reference marker of mechanochromic material remains fixed to a tooth for the duration of several repositioning steps or even the entire treatment. This second embodiment is advantageous from an economical point of view. It goes without saying that a plurality of reference markers of mechanochromic material can be placed at different sites of the patient's tooth or gingiva arrangement.

Another aspect of the invention relates to a non-transitory computer readable medium, having a computer program logic recorded thereon which when executed by a processor causes a device to:
receive data corresponding to at least one image taken by a portable device for image acquisition, wherein the at least one image depicts a removable orthodontic appliance and at least one of
   a reference marker applied thereto; and
   a reference marker applied to a tooth or gingiva area of a patient;
compare the received data corresponding to the at least one image to reference data corresponding to a reference image.

By means of such a non-transitory computer readable medium, the method as described above can be facilitated. In particular, when run on a user's device, important steps of the monitoring activities can be performed by the patients themselves, hence reducing the number of dentist's appointments and increasing patient motivation and compliance.

It is preferred that the non-transitory computer readable medium as described above is such that when the computer program logic recorded thereon is executed by a processor it:
receives data corresponding to a stress level in the removable orthodontic appliances, wherein a piezochromic material, a piezochromic coating or a piezochromic ink embedded in a matrix is used as a pressure sensor or differential pressure sensor in a removable orthodontic appliance; and
measures or monitors the said stress level

It is preferred that the computer program logic recorded on the computer readable medium as described above when executed by a processor additionally causes the device to
assess stress relaxation in the removable orthodontic appliance based on at least one of a positioning and an optical property of the reference marker.

It is preferred that the reference marker is configured as described above, in particular comprises a mechanochromic material as described above.

It is preferred that the computer program logic recorded on the computer readable medium as described above when executed by a processor additionally causes the device to
super-impose the acquired images and compare the acquired images to extract information about at least one of
   a stress relaxation in the removable orthodontic appliance; and
   a displacement of the patient's teeth.

It is further preferred that the computer program logic recorded on the computer readable medium when executed by a processor additionally causes the device to:
display the results of the image comparison for monitoring an orthodontic treatment.

Another aspect of the invention relates to a system for monitoring the treatment progress of a teeth alignment process with removable orthodontic appliances comprising:
a set of removable orthodontic appliances, having at least one reference markers applied thereto, preferably a reference marker as described above;
a non-transitory computer readable medium, having a computer program logic recorded thereon which when executed by a processor causes a device to:
receive data corresponding to at least one image from a portable device for image acquisition;
compare the received data corresponding to an image to reference data corresponding to a reference image.

It is preferred that the removable orthodontic appliances are configured as described above. It is further preferred that the non-transitory computer readable medium, having a computer program logic recorded thereon is configured as described above.

For example one embodiment, a non-transitory computer readable medium can have a program logic recorded thereon such that the computer program logic recorded when executed by a processor:
receives data corresponding to a stress level in the removable orthodontic appliances, wherein a piezochromic material, a piezochromic coating or a piezochromic ink embedded in a matrix is used as a pressure sensor or differential pressure sensor in the removable orthodontic appliances.
measures or monitors the said stress level.

This embodiment can be provided as an alternative to the non-transitory computer readable medium as described above or can provide the listed features on top of those listed for the non-transitory computer readable medium as described above.

It is preferred that, the computer program logic of the system, when executed by a processor, causes the device to perform the actions as described above. For example, it is preferred that the computer program logic when executed by a processor additionally causes the device to assess stress relaxation in the removable orthodontic appliance based on at least one of a positioning and an optical property of the reference points.

Also, it is preferred that the computer program logic of the system when executed by a processor causes the device to
super-impose the acquired images and compare the images to extract information about at least one of
   a stress relaxation in the orthodontic appliances; and
   a displacement of the patient's teeth.

Further, it is preferred that the system's program logic when executed by a processor additionally causes the device to display the results of the image comparison for monitoring an orthodontic treatment.

In one embodiment, the computer readable medium is configured such that the computer program logic recorded thereon when executed by a processor additionally causes the device to share the data with a different electronic device for data processing. For example, the data could be shared with a dentist's computer or with another professional's device. Sharing could occur via WiFi, Bluetooth and/or a computer network, such as the internet.

It is particularly preferred that the device which is driven by the computer program logic recorded on a computer readable medium as described above is a smartphone, a tablet or patient's smartphone, a patient's tablet or any other portable device.

It is further preferred that the computer program logic recorded on the computer readable medium when executed by a processor additionally causes the device to remind a patient to retrieve an image depicting a removable orthodontic appliance and at least one of a reference marker applied thereto and a reference marker applied to a tooth/gingiva area of a patient on a regular basis. Particularly preferably the patient is reminded every day to wear the removable tooth aligner and to monitor the progress of the treatment in order to enhance patient compliance.

A further aspect of the invention relates to a set for aligning teeth with a removable orthodontic appliance according as described above, wherein the set additionally comprises a non-transitory computer readable medium as described above. The set of aligner(s) and mechano-chromic reference marker(s) is advantageously combined with such computer readable medium because it supports the patient's and the dentist's decision making on the next treatment steps.

The system as described above may additionally comprise a magnifying lens, wherein prior to the image acquisition the magnifying lens can be attached to a camera of the portable device for image acquisition in order to acquire at least on high resolution image of removable orthodontic appliances mounted on dental arches, such that stress relaxation in the orthodontic appliances can be visualized.

The system as described above may additionally comprise at least one lens for use in image acquisition, wherein the at least one lens is configured to visualize residual stresses in the orthodontic appliances based on birefringence.

1. A removable orthodontic appliance, having at least one reference marker applied thereto, wherein the at least one reference marker comprises a mechanochromic material, preferably a piezochromic material.

2. Removable orthodontic appliance according to claim 1 wherein the at least one reference marker is a graphic printed element.

3. Removable orthodontic appliance according to claim 2 wherein the graphic printed element is designed such that detection by eye is rendered possible.

4. Removable orthodontic appliance according to claim 2 wherein the graphic printed element is only visible under light other than white light.

5. Removable orthodontic appliance according to claim 1, where the mechanochromic material is an organic chromophore dispersed in polymer matrix, an inorganic solid, or a liquid crystal element.

6. Removable orthodontic appliance according to claim 1, wherein the orthodontic appliance has a calibration device mounted thereto which is configured to standardize the displacement measurements.

7. Removable orthodontic appliance according to claim 6, wherein the calibration device can be adjusted by a user to monitor changes of a tooth position.

8. Removable orthodontic appliance according to claim 1 having at least one element of the group of sensors and actuators attached or included thereto, wherein the element is configured to communicate with a patient's portable device.

9. A set for aligning teeth with a removable orthodontic appliance, wherein the set comprises:
a plurality of removable orthodontic appliances having at least one reference marker applied thereto, wherein the at least one reference marker comprises a mechanochromic material; or
at least one removable orthodontic appliance and at least one reference marker comprising a mechanochromic material.

10. A non-transitory computer readable medium, having a computer program logic recorded thereon which when executed by a processor causes a device to:
receive data corresponding to at least one image taken by a portable device for image acquisition, wherein the at least one image depicts a removable orthodontic appliance and at least one of
a reference marker applied thereto; and
a reference marker applied to a tooth or gingiva area of a patient.
compare the received data corresponding to the at least one image to reference data corresponding to a reference image.

11. Non-transitory computer readable medium according to claim 10, wherein the computer program logic recorded thereon when executed by a processor, causes the device to:
receive data corresponding to a stress level in the removable orthodontic appliances, wherein a piezochromic material, a piezochromic coating or a piezochromic ink embedded in a matrix is used as a pressure sensor or differential pressure sensor in a removable orthodontic appliance; and
measure or monitor the said stress level.

12. Computer readable medium according to claim 10, wherein the computer program logic recorded thereon when executed by a processor additionally causes the device to
assess stress relaxation in the removable orthodontic appliance based on at least one of a positioning and an optical property of the reference marker.

13. Computer readable medium according to claim 10, wherein the computer program logic recorded thereon when executed by a processor additionally causes the device to
super-impose the acquired images and compare the acquired images to extract information about at least one of
a stress relaxation in the removable orthodontic appliance; and
a displacement of the patient's teeth.

14. Computer readable medium according to claim 10, wherein the computer program logic recorded thereon when executed by a processor additionally causes the device to
display the results of the image comparison for monitoring an orthodontic treatment.

15. Computer readable medium according to claim 10, wherein the computer program logic recorded thereon when executed by a processor additionally causes the device to:
super-impose the acquired images and compare the images to extract information about at least one of
a stress relaxation in the orthodontic appliances; and
a displacement of the patient's teeth.

16. Computer readable medium according to claim 10, wherein the computer program logic recorded thereon when executed by a processor additionally causes the device to display the results of the image comparison for monitoring an orthodontic treatment.

17. Computer readable medium according to claim 10, wherein the computer program logic recorded thereon when executed by a processor additionally causes the device to share the data with a different electronic device for data processing.

18. Computer readable medium according to claim 10, wherein the device is a patient's smartphone, a patient's tablet computer or any other portable device.

19. Computer readable medium according to claim 10, wherein the computer program logic recorded thereon when executed by a processor additionally causes the device to remind a patient to retrieve an image depicting a removable orthodontic appliance and at least one of
a reference marker applied thereto; and
a reference marker applied to a tooth or gingiva area of a patient;
on a regular basis.

20. A set for aligning teeth with a removable orthodontic appliance according to claim 9, wherein the set additionally comprises a non-transitory computer readable medium having a computer program logic recorded thereon which when executed by a processor causes a device to:
receive data corresponding to at least one image taken by a portable device for image acquisition, wherein the at least one image depicts a removable orthodontic appliance and at least one of
a reference marker applied thereto; and
a reference marker applied to a tooth or gingiva area of a patient.
compare the received data corresponding to the at least one image to reference data corresponding to a reference image.
